SEQUENCE LISTING (1 sequence)

SEQ ID NO: 1 - protein, 10 aa, synthetic construct
TTDPSFLGRY

(19) United States
(12) Patent Application Publication
(10) Pub. No.: US 2024/0358753 A1
(43) Pub. Date: Oct. 31, 2024

(54) METHODS FOR DEVELOPING CD3+CD8+ CELLS AGAINST MULTIPLE VIRAL EPITOPES FOR TREATMENT OF VIRAL INFECTIONS INCLUDING VARIANTS EVOLVING TO ESCAPE PREVIOUS IMMUNITY

(71) Applicant: Tevogen Bio Inc., Metuchen, NJ (US)

(72) Inventor: Ryan SAADI, Metuchen, NJ (US)

(21) Appl. No.: 18/291,142

(22) PCT Filed: Jul. 29, 2022
(86) PCT No.: PCT/US2022/038749

§ 371 Date: Jan. 22, 2024

Related U.S. Application Data
(60) Provisional application No. 63/227,690, filed on Jul. 30, 2021.

(51) Int. Cl. A61K 35/17; A61K 39/00; C07K 14/005; C12N 5/0783

(52) U.S. Cl. CPC A61K 35/17; A61K 39/4611; A61K 39/464838; C07K 14/005; C12N 5/0638; C12N 2770/20033

(57) ABSTRACT

Disclosed herein are methods of preparing peptide specific cytotoxic T cells (CTLs) against an emerged strain of a virus. The methods include providing an initial peptide composition specific to a prior strain of a virus, against which prior CTLs were sensitized, wherein the prior CTLs have reduced efficacy against an emerged strain of a virus relative to their efficacy against the prior strain of the virus; identifying an immunodominant peptide against a prior strain of a virus in an initial peptide composition; reducing the proportion of an immunodominant peptide in an initial peptide composition to yield an immunodominant-peptide-diluted peptide composition; and sensitizing mononuclear cells with an immunodominant-peptide-diluted peptide composition, thereby producing expansion of the peptide specific CTLs against an emerged strain of a virus. The virus can be SARS-COV2 (COVID-19) and the emerged strain can be Delta or Omicron BA.2.75.

### CROSS-REFERENCE TO RELATED APPLICATIONS

This application claims the benefit of U.S. Provisional Patent Application 63/227,690, filed Jul. 30, 2021, the entirety of which is hereby incorporated by reference herein.

### BACKGROUND

Infectious diseases were responsible for the largest global burden of premature death and disability until the end of the twentieth century, when that distinction passed to noncommunicable diseases. Of infectious disease, viral infections are a major worldwide health issue with capacity for emerged pandemic threat.

SARS-COV-2 (COVID-19) has resulted in over 4 million deaths worldwide. While vaccination has decreased mortality, there remains a need for curative therapies for active infections. Uncertainties regarding the duration of post-vaccination immunity and the rapidity of mutational evolution by this virus suggest that it is unwise to rely on preventative measures alone.

Humoral and cellular immunity provide selective pressure for the emergence of variant strains which have eliminated target epitopes. Elimination of immunodominant epitopes provides the strongest advantage to newly emerging strains and, consequently, immunodominant epitopes would be expected to be preferentially eliminated compared to subdominant epitopes in emerging variants. Immunologic treatments for SARS-COV-2 need to be continuously reassessed as new sequence information becomes available.

Although SARS-COV-2 has gripped the attention of the global health community since late 2019, the elimination of immunodominant epitopes and the emergence of new variants is a phenomenon present in numerous viral infectious diseases, in humans and in other animal species.

Accordingly, techniques for reassessment of immunologic treatments for many viruses are needed to better defend human and animal health against emerging variants of infectious viruses.

### SUMMARY

This disclosure relates to methods of preparing peptide specific cytotoxic T cells (CTLs) against an emerged strain of a virus.

The methods can comprise providing an initial peptide composition specific to a prior strain of the virus, against which prior CTLs were sensitized, wherein the prior CTLs have reduced efficacy against the emerged strain of the virus relative to their efficacy against the prior strain of the virus.

In some embodiments, the prior CTLs can have reduced efficacy against the emerged strain of the virus due to loss of an immunodominant peptide target in the emerged strain relative to the prior strain.

The methods can comprise identifying an immunodominant peptide against the prior strain of the virus in the initial peptide composition.

The methods can comprise reducing the proportion of the immunodominant peptide in the initial peptide composition to yield an immunodominant-peptide-diluted peptide composition. In some embodiments, the proportion of the immunodominant peptide in the immunodominant-peptide-diluted peptide composition can be at least 30% or less than the proportion of the immunodominant peptide in the initial peptide composition. In some embodiments, the immunodominant peptide can be essentially absent from the immunodominant-peptide-diluted peptide composition.

The methods can comprise sensitizing mononuclear cells with the immunodominant-peptide-diluted peptide composition, thereby producing expansion of the peptide specific CTLs against the emerged strain of the virus.

The methods can be used to prepare peptide specific CTLs against any emerged strain of any virus. In some embodiments, the virus can be SARS-COV-2 (COVID-19). In some embodiments, the emerged strain can be Delta or Omicron BA.2.75.

The methods can be used with initial peptide compositions restricted against any HLA allele. In some embodiments, the initial peptide composition can comprise one or more peptides restricted against an HLA-A1 allele. In some embodiments, the initial peptide composition can comprise one or more peptides restricted against an HLA-A2 allele. In some embodiments, the initial peptide composition can comprise one or more peptides restricted against an HLA-B7 allele. In some embodiments, the initial peptide composition can comprise one or more peptides restricted against an HLA-B40 allele. In some embodiments, the initial peptide composition can comprise one or more peptides restricted against an HLA-Cw7 allele. In some embodiments, the initial peptide composition can comprise a combination of peptides binding to any one or more of HLA-A1, A2, B7, B40, Cw7 alleles.

The disclosure also relates to methods of treating an emerged strain of a virus. The methods can comprise administering to a subject in need thereof an effective amount of peptide specific CTLs prepared according to the methods described herein. In some embodiments, in methods of treatment, the virus against which the peptide specific CTLs are prepared can be COVID-19. In some embodiments, in methods of treatment, the emerged strain can be Delta or Omicron BA.2.75.

In some embodiments, methods of treatment can further comprise administering one or more pre-medications prior to administering the CTLs.

In some embodiments, methods of treatment can further comprise administering one or more antiviral formulations prior to, concurrently with, or subsequently to administering the CTLs.

The disclosure also relates to pharmaceutical compositions comprising peptide specific CTLs prepared according to the methods described herein. In some embodiments, in the pharmaceutical compositions, the virus against which the peptide specific CTLs are prepared can be COVID-19. In some embodiments, in the pharmaceutical compositions, the emerged strain can be Delta or Omicron BA.2.75.

In some embodiments, in the pharmaceutical compositions, the proportion of the immunodominant peptide in the immunodominant-peptide-diluted peptide composition can be at least 30% or less than the proportion of the immunodominant peptide in the initial peptide composition. In some embodiments, in the pharmaceutical compositions, the immunodominant peptide can be essentially absent from the immunodominant-peptide-diluted peptide composition.

### BRIEF DESCRIPTION OF THE DRAWINGS

FIG. 1A shows tetramer staining assay results for a composition comprising CD8+ CTLs recognizing peptides derived from COVID-19 gene/ORF products in an HLA restricted manner, as described in Example 1.

FIG. 1B shows cytotoxicity assay results for the composition comprising CD8+ CTLs recognizing peptides derived from COVID-19 gene/ORF products in an HLA restricted manner described in Example 1.

FIG. 2A presents a pie chart of immunodominance as determined by tetramer staining assay of CTLs generated from apheresis products from two donors for a composition containing seven peptides binding to HLA-A*02:01, as described in Example 1.

FIG. 2B presents a pie chart of immunodominance as determined by tetramer staining assay of CTLs generated from apheresis products from one donor for a composition containing eight peptides binding to HLA-A*01:01, as described in Example 1.

### DETAILED DESCRIPTION

This disclosure relates to the preparation of peptide-specific cytotoxic T lymphocytes (CTLs) that can be used for immunological treatment of subjects infected by an emerged strain of a virus. The CTLs can be prepared by providing an initial peptide composition specific to a prior strain of the virus, against which prior CTLs were sensitized, wherein the prior CTLs have reduced efficacy against the emerged strain of the virus relative to their efficacy against the prior strain of the virus; identifying an immunodominant peptide against the prior strain of the virus in the initial peptide composition; reducing the proportion of the immunodominant peptide in the initial peptide composition, to yield an immunodominant-peptide-diluted peptide composition; and sensitizing mononuclear cells with the immunodominant-peptide-diluted peptide composition, thereby producing expansion of the peptide specific CTLs against the emerged strain of the virus.

All publications and patents cited in this disclosure are incorporated by reference in their entirety. To the extent, the material incorporated by reference contradicts or is inconsistent with this specification, the specification will supersede any such material. The citation of any references herein is not an admission that such references are prior art to the present disclosure. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise.

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The term “subject” as used herein refers to any animal, such as any mammal, including but not limited to, humans, non-human primates, rodents, mammals commonly kept as pets (e.g., dogs and cats, among others), livestock (e.g., cattle, sheep, goats, pigs, horses, and camels, among others) and the like. In some embodiments, the mammal is a mouse. In some embodiments, the mammal is a human.

Additional description of the methods and guidance for the practice of the methods are provided herein.

### Viruses and Strains

Numerous viruses pose a burden on human or animal health, and undergo frequent emergence of new strains with epitopes distinct enough from those of prior strains to evade humoral and cellular immunity developed against those prior strains. Examples of such viruses include, but are not limited to, SARS-COV-2 (COVID-19), influenza, parainfluenza, respiratory syncytial virus (RSV), metapneumovirus, Hepatitis B virus (HBV), Epstein-Barr virus (EBV), cytomegalovirus (CMV), BK virus (BKV), John Cunningham virus (JCV), human herpesvirus (HHV), and adenovirus.

In some embodiments, the virus can be SARS-COV-2 (COVID-19).

As used herein, a prior strain is a strain of the virus previously subjected to immunologic treatments with CTLs. Specifically, an initial peptide composition was derived from the prior strain, prior CTLs were sensitized against the initial peptide composition, and the prior CTLs had efficacy in treating an infection of a mammal by the prior strain.

As used herein, an emerged strain is a strain of the virus differing from the prior strain, wherein the prior CTLs have reduced efficacy against the emerged strain of the virus relative to their efficacy against the prior strain of the virus.

In some embodiments, if the virus is COVID-19, the emerged strain can be Delta or Omicron BA.2.75.

### Initial Peptide Compositions and Prior CTLs

The method can comprise providing an initial peptide composition specific to a prior strain of the virus, against which prior CTLs were sensitized, wherein the prior CTLs have reduced efficacy against the emerged strain of the virus relative to their efficacy against the prior strain of the virus.

The initial peptide composition can comprise about 2, about 3, about 4, about 5, about 6, about 7, about 8, about 9, about 10, about 11, about 12, about 13, about 14, about 15, about 16, about 17, about 18, about 19, about 20, about 1 to about 5, about 1 to about 10, about 1 to about 15, about 1 to about 20, about 2 to about 5, about 2 to about 10, about 2 to about 15, about 2 to about 20, about 5 to about 10, about 5 to about 15, about 5 to about 20, about 10 to about 20, or about 15 to about 20 peptides. The specific peptides of the initial peptide composition are distinct for each virus and each prior strain. Any list of peptides set forth in this application is solely exemplary and non-limiting, because any such list is both not exhaustive and not fixed, and will continue as a dynamic, open-ended list.

The peptides can be chosen from those that bind to an HLA restriction element of interest. The HLA restriction element of interest may be selected from three classical HLA-I genes expressed in all nucleated cells in humans: HLA-A, HLA-B, and HLA-C. HLA-I molecules present peptides derived from intracellular proteins. The intracellular antigen presentation pathway may involve cleavage of viral proteins in the cytosol by proteasomes, translocation to the endoplasmic reticulum (ER) lumen, trimming by ER-resident aminopeptidases, loading onto HLA, and presentation at the cell surface. HLA-II genes (HLA-DR, HLA-DP and HLA-DQ) are constitutively expressed in only a subset of cells specialized for antigen presentation, such as dendritic cells, B cells, and macrophages, but expression can also be induced in additional cell types, e.g. in response to cytokine stimulation. HLA-II molecules present peptides derived from extracellular proteins taken into cells via endocytosis and phagocytosis, and intracellular proteins that access the HLA-II processing pathway via autophagy.

HLA-I molecules typically bind peptides of 8-12 amino acids (aa) in length. The HLA-I peptide-binding cleft is closed at both N- and C-terminal ends, and optimal length preferences are often biased towards binding of ˜9-mer peptides. For most HLA-I alleles the length preferences differ between alleles. High affinity ligands for a given HLA allele usually share a common amino acid motif with relatively strict preferences in anchor positions (for HLA-I usually the second (P2) and last (PΩ), for HLA-II-P1, P4, P6 and P9), which form specific interactions with residues of corresponding HLA binding pockets. The HLA locus is the most polymorphic in the human genome with tens of thousands alleles described to date. HLA variants that differ in peptide-contacting residues differ in the repertoire of peptides they present. The diversity of HLA alleles in the population is an important evolutionary mechanism for defense against diverse pathogens, e.g. rapidly mutating viruses, newly emerged viruses and viral strains, and the like. HLA alleles may be associated with the severity and outcomes of viral infections. For example, the HLA-C* 15:02 allele is associated with protection against SARS-COV-1, and HLA-B57 is highly associated with efficient HIV-1 control and long-term non-progressive infection in the absence of antiretroviral therapy.

The peptides of the initial peptide composition can originate from any virus. The peptides can be found empirically, by bioinformatics techniques, or from publicly-available sources. For example, T cell epitopes have been identified, collected, and reported for a wide range of viruses in the Immune Epitope Database and Analysis Resource (IEDB).

In some embodiments, the initial peptide composition can comprise one or more peptides restricted against an HLA-A1 allele.

Additionally or alternatively, in some embodiments, the initial peptide composition can comprise one or more peptides restricted against an HLA-A2 allele.

Alternatively or in addition, in some embodiments, the initial peptide composition can comprise one or more peptides restricted against an HLA-B7 allele.

Additionally or alternatively, in some embodiments, the initial peptide composition can comprise one or more peptides restricted against an HLA-B40 allele.

Alternatively or additionally, in some embodiments, the initial peptide composition can comprise one or more peptides restricted against an HLA-Cw7 allele.

As should be apparent, in some embodiments, the initial peptide composition can comprise a combination of peptides, each peptide binding to any one or more of HLA-A1, -A2, -B7, -B40, or -Cw7 alleles.

### Identification of Immunodominant Peptides

The method can also comprise identifying an immunodominant peptide against a prior strain of a virus in an initial peptide composition. For example, identifying can be performed by tetramer assays, interferon production assays, cytotoxicity assays, or other known techniques in which T cells with specificity for an individual peptide can be found within a T cell population.

After individually assaying each peptide in an initial peptide composition against a T cell population containing prior CTLs, an immunodominant peptide(s) can be identified based on one or more conditions. The peptide with the highest score in the tetramer assay, interferon production assay, cytotoxicity assay, or other assay can be identified as an immunodominant peptide. This approach can be modified by requiring the highest score to be greater than a threshold level, such as about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, or about 10%. In this modified approach, an initial peptide composition might be deemed to have no immunodominant peptides if the highest score among all peptides is below the threshold level.

In another approach, any score greater than a threshold level can be deemed sufficient to identify a peptide as an immunodominant peptide. In this approach, an initial peptide composition might be deemed to have zero, one, two, or more immunodominant peptides.

Desirably, the conditions for identifying an immunodominant peptide in an initial peptide composition should be chosen such that at least one peptide will be identified as an immunodominant peptide, given that the prior CTLs sensitized against an initial peptide compositions have been found to have reduced efficacy against an emerged strain of a virus relative to their efficacy against the prior strain of the virus. Though not to be bound by theory, the prior CTLs may have reduced efficacy due to their disproportionate recognition of an immunodominant peptide which has been lost in the emerged strain. Modification of an initial peptide composition is therefore warranted.

If two or more peptides in an initial peptide composition are identified as immunodominant, the further steps below can be performed on one, some, or all of the immunodominant peptides.

### Reduction of Proportion of Immunodominant Peptide in Peptide Compositions

The method also can comprise reducing the proportion of an immunodominant peptide in an initial peptide composition, to yield an immunodominant-peptide-diluted peptide composition.

Reduction can involve the removal of peptide molecules from an initial peptide composition, or the production of a composition with controlled proportions of the same peptides included in an initial peptide composition.

The proportion of an immunodominant peptide in an immunodominant-peptide-diluted peptide composition can be reduced to any desired level, such as to about 50% or less, about 49% or less, about 48% or less, about 47% or less, about 46% or less, about 45% or less, about 44% or less, about 43% or less, about 42% or less, about 41% or less, about 40% or less, about 39% or less, about 38% or less, about 37% or less, about 36% or less, about 35% or less, about 34% or less, about 33% or less, about 32% or less, about 31% or less, about 30% or less, about 29% or less, about 28% or less, about 27% or less, about 26% or less, about 25% or less, about 24% or less, about 23% or less, about 22% or less, about 21% or less, about 20% or less, about 19% or less, about 18% or less, about 17% or less, about 16% or less, about 15% or less, about 14% or less, about 13% or less, about 12% or less, about 11% or less, about 10% or less, about 9% or less, about 8% or less, about 7% or less, about 6% or less, about 5% or less, about 4% or less, about 3% or less, about 2% or less, or about 1% or less than the proportion of an immunodominant peptide in an initial peptide composition.

In some embodiments, the proportion of an immunodominant peptide in an immunodominant-peptide-diluted peptide composition can be reduced to at least 30% or less than the proportion of the immunodominant peptide in the initial peptide composition.

In some embodiments, an immunodominant peptide can be essentially absent (i.e., if present, is present at a level undetectable in the same assay used to identify an immunodominant peptide in an initial peptide composition) from an immunodominant-peptide-diluted peptide composition.

### Production of Peptide Specific CTLs Against Emerged Strain of Virus

The method can also comprise sensitizing mononuclear cells with an immunodominant-peptide-diluted peptide composition, thereby producing expansion of peptide specific CTLs against an emerged strain of a virus.

In one exemplary approach, lymphocytes (a type of mononuclear cells) undergo three in vitro stimulation-expansion cycles to produce the final CTL products used in the methods of treatment described herein. Each of these three stimulation-expansion cycles has a different purpose within the overall production process, and each therefore follows a distinct procedure. Optionally, a fourth restimulation may be performed. The fourth restimulation may be performed (1) for products which fall slightly short of meeting release criteria when it is anticipated that an additional round of stimulation and expansion will allow the product to meet these criteria or (2) if additional cell expansion is desired and it is thought that an additional round of stimulation and expansion will likely significantly increase the number of treatment doses which can be obtained from that batch. Any such optional fourth restimulation may be performed following the identical process for the third stimulation.

In some embodiments, mononuclear cells from healthy volunteer donors can be separated by elutriation into lymphocyte and monocyte fractions. Lymphocytes can be stimulated with the immunodominant-peptide-diluted peptide composition. Viral derived products need not be used.

In the first stimulation, a subset of the collected monocytes can be treated so as to induce their maturation into dendritic cells. Dendritic cells can be pulsed with one or more viral-specific peptides and co-cultured with lymphocytes for 7 days. The second and third stimulations can use monocytes to present the peptides and again can allow 7-12 days for stimulated lymphocytes to grow/expand. The second stimulation also can include an enrichment step which helps to select for peptide specific CTLs (due to preferential adherence of T cells recognizing the pulse peptides to an adherent monocyte layer) and reduces the content of other non-specific ‘bystander’ lymphocytes or other immune cells from the donor. The third stimulation again can use monocytes and peptides, but need not repeat this selection step.

Most of the procedures described herein can be performed in RPMI-1640 with 10% heat inactivated AB serum. The amount of AB serum in the media may be reduced to about 9%, about 8%, about 7%, about 6%, about 5%, about 4%, about 3%, about 2%, or about 1%. Serum-free media and autologous plasma may be used as alternatives. This is referred to as “complete media” or CM. Any suitable media can be used, as determined by one of skill in the art, such as AIM V or other serum free media preparations alone, with about 10% or lower concentrations of pooled serum or autologous serum or plasma, RPMI-1640 with serum substitutes with or without lower concentrations of pooled serum or autologous serum or plasma.

In some embodiments, a first stimulation, referred to as the Initial In Vitro Sensitization, lymphocytes can be stimulated with an immunodominant-peptide-diluted peptide composition as a pool, not as individual peptides.

For an Initial In Vitro Sensitization, dendritic cells can be used as antigen presenting cells. These cells can be prepared from elutriated monocytes. Fresh or freshly thawed monocytes can be enriched by adherence to plastic. A suitable number of monocytes can be resuspended in media and then cells can be transferred to a tissue culture plate. The cells can be then incubated for a suitable time (e.g., at least 60 minutes, at least 90 minutes, at least 120 minutes) to allow the monocytes to adhere to the culture plate. After incubation, the supernatant can be removed from the culture plate. Adherent cells may then then be cultured with GM-CSF and IL-4 for a suitable time (e.g., 24 hours), at which point maturation cytokines (e.g., TNF-alpha, IL-1 beta, IL-6, and/or prostaglandin E2) may be added. After another 24 hours of culture, the dendritic cells detach and can be ready for harvest by aspiration and centrifugation of the media. The duration of culture in maturation cytokines may, if necessary, be extended beyond 24 hours to about 30 hours, about 36 hours, about 42 hours, or up to about 48 hours.

Following their harvest, dendritic cells can be pulsed with peptides (e.g., 2 μg/ml each) for a suitable length of time (e.g., for about 60, 75, 90, or 120 minutes) and co-cultured thereafter with lymphocytes in tissue culture flasks (e.g., 75 cm²) in media (e.g., CM). The ratio of lymphocytes to dendritic cells in culture may be about 15:1, about 16:1, about 17:1, about 18:1, about 19:1, about 20:1, about 21:1, about 22:1, about 23:1, about 24:1, or about 25:1. Lymphocytes (e.g., a total of 80×10⁶, 100×10⁶, 120×10⁶) can be then added to each flask. This can be considered day 0 of the CTL culture process. Cultures can be left undisturbed after this stimulation for 7 days.

In the second stimulation, enrichment and subsequent expansion of viral specific CTLs occurs. Seven days after the initial sensitization, CTLs can be re-stimulated as part of an enrichment step which helps to select for peptide specific CTL and reduces the content of other non-specific ‘bystander’ lymphocytes or other immune cells from the donor. Enrichment can be based on preferential adherence of peptide specific CTLs to a monocyte layer which has been pulsed with the peptides used for the initial sensitization. CTLs recognizing any of the peptides as presented by the appropriate HLA allele will preferentially adhere to the monocyte plus peptide layer through creation of an immunologic synapse in contrast to ‘bystander’ lymphocytes, which can be gently washed away. While some ‘bystander’ lymphocytes may non-specifically adhere, this typically allows for an approximately 10-fold or greater enrichment of peptide-specific CTLs versus the starting material. To perform this enrichment step, monocytes (e.g., 10×10⁶) can be added to tissue culture plates. Peptides can be added (e.g., at a final concentration of 2 μg/ml each) and allowed to incubate with the monocytes (e.g., for about 90 minutes). Lymphocytes (e.g., about 60×10⁶, about 70×10⁶, about 80×10⁶, about 90×10⁶, about 100×10⁶, about 110×10⁶, or about 120×10⁶, typically the contents of one of the 75 cm² tissue culture flasks) can be then added to the wells. ‘Bystander’ lymphocytes can be removed from the wells by gentle washing with PBS after an appropriate length of time (e.g., about 5, about 7.5, about 10, or about 12 minutes). Adherent lymphocytes can be allowed to remain in contact with peptide pulsed monocytes overnight to complete the activation/restimulation process. The following day, lymphocytes can be removed from monocyte layers. The dislodged, adherent lymphocytes can be transferred to tissue culture flasks in media with recombinant human IL-2 (e.g., at a concentration of 50 U/ml). IL-2 can be added over time (e.g., 50 U/ml every 48 hours). Media can be changed if/when flasks show conversion to a more orange/yellow color. Cells can then be cultured for a total of 7 days following the second stimulation. Enrichment on the monocyte layer as part of this second stimulation is desirable and can provide an advantage over prior methods. Without wishing to be bound by theory, it is believed that the enrichment on the monocyte layer is responsible for the significantly higher purity level amongst total T cells that has not been achieved prior to the present invention.

A third stimulation can be performed to further expand the viral peptide specific CTLs. The enrichment step performed as part of the second stimulation need not be repeated as part of the third stimulation. However, if assessment of the percentage of viral-peptide reactive lymphocytes (as measured by intracellular cytokine assay or tetramer assay) is below a certain limit (e.g., about 12-18%, about 12%, about 13%, about 14%, about 15%, about 16%, about 17% or about 18%) within a day of the planned third stimulation, the procedure for the second stimulation can be repeated in lieu of the usual procedure for the third stimulation. Of the entire CTL production process, the enrichment step involves the most manipulation and can be the point most vulnerable to introduce contamination, and thus it can be desirable to avoid repeating this step more than once to the extent possible. Further enrichment of the percentage of viral peptide-specific CTLs can be anticipated after the third stimulation, even without repeating the enrichment step. This reflects the fact that stimulated cells will grow in IL-2 containing media whereas unstimulated ‘bystander’ cells will not and that, over time, unstimulated ‘bystander’ cells will die off in culture leading to a more enriched product. By setting the above threshold for when the enrichment step from the second stimulation may be repeated, it is anticipated that it will be repeated infrequently, and only when essential to the manufacturing process.

After seven days of culture in the tissue culture flask (day 14 of CTL stimulation/culture overall), cells can be counted and restimulated with monocytes and peptide in G-Rex flasks. The lymphocyte: monocyte ratio can be between about 4:1 and about 5:1, about 4:1, about 4.1:1, about 4.2:1, about 4.3:1, about 4.4:1, about 4.5:1, about 4.6:1, about 4.7:1, about 4.8:1, about 4.9:1, or about 5:1. Each peptide can be again added at a concentration of about 2 μg/ml. Lymphocytes (e.g., 15×10⁶) can be added to each G-Rex10 flask for this secondary restimulation. Sensitization can be performed in media (e.g., 40 ml of complete media) with IL-2 (e.g., 50 U/ml).

Following the third stimulation, CTLs can be again cultured (e.g., for 7 days). Media and IL-2 change can be performed every 3-4 days depending on when media color change is observed.

Following conclusion of this 21 day period of stimulation and expansion, CTLs can be assessed as to whether they meet the necessary criteria and, if so, harvested for cryopreservation (e.g., within 24 hours thereafter). Optionally, in some circumstances a fourth stimulation can be performed following the guidelines for the third stimulation. This will typically be performed when further cell expansion is deemed desirable to increase the number of doses of CTLs being generated or if products fall slightly short of release criteria and it is thought that an additional round of stimulation/expansion will allow the product to meet all criteria. Restimulation steps can be performed at 6-10 day intervals (e.g., 6 day, 7 day, 8 day, 9 day, 10 day intervals), though these steps can occur up to one day earlier or later than this typical 7-day interval, if necessary.

After completion of the three in vitro stimulation-expansion cycles, products can be then screened for appropriate cellular content, function, viability and sterility.

Appropriate cellular content means at least about 20% (e.g., at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%) of the cells will respond to viral peptides based on intracellular cytokine (ICC) staining, or tetrameter binding, and that the content of naïve T cells, monocytes, and NK cells in the product can be less than about 2.5% (e.g., about 2.4%, about 2.3%, about 2.2. %, about 2.1%, about 2.0%, about 1.9%, about 1.8%, about 1.7%, about 1.6%, about 1.5%, about 1.4%, about 1.3%, about 1.2%, about 1.1%, about 1.0%, about 0.9%, about 0.8%, about 0.7%, about 0.6%, about 0.5%, about 0.4%, about 0.3%, about 0.2%, or about 0.1%).

Function can be based on about 40% cytolytic activity of the CTL toward peptide pulsed targets at an effector: target ratio of about 40:1.

Viability should exceed 70%, for example viability of about 75%, about 80%, about 85%, about 90%, about 95%, or about 99% can be appropriate.

Sterility may be assessed through routine and fungal cultures, as well as assays for mycoplasma and endotoxin. CTLs may be cryopreserved in cryobags at any desired concentration, for example a concentration of about 2×10⁶ per milliliter, and stored for later use in the methods disclosed herein.

Methods of Treating an Emerged Strain of a Virus with Peptide Specific CTLs

The methods can further comprise administering to a subject an effective amount of peptide specific CTLs prepared as described above.

In some embodiments, the methods can involve administering, by intravenous infusion, an effective amount of peptide specific CTLs to a subject in need thereof. The peptide specific CTLs may be administered by intravenous delivery to the subject, for example, by administration through a central line, midline, or peripheral IV.

In some embodiments, prior to administration of the peptide specific CTLs, subjects can undergo full HLA typing and then be treated with an appropriate CTL.

In some embodiments, prior to administration of the peptide specific CTLs, subjects can have their blood tested for rapid, low resolution human leukocyte antigens (HLA) typing with high resolution PCR SSP supplementation to determine if they have a potentially appropriate HLA antigen for the treatment (HLA-A1, -A2, -B7, -B40, -Cw7). The high-resolution supplementation can provide evidence that the subjects are HLA-A*01:01, -A*02:01, -B*07:02, -B*40:01, -C*07:02 and thus match the CTL for one or more alleles.

Prior to administration of the peptide specific CTLs, pre-medications can be administered. In some embodiments, subjects can receive pre-medications, such as diphenhydramine and acetaminophen. The diphenhydramine dose can be about 15, about 20, about 25, or about 30 mg. The acetaminophen dose can be about 500, about 550, about 600, about 650, about 700, or about 750 mg.

Subjects can also be treated with one or more antiviral formulations (e.g., formulations containing remdesivir or other standard of care pharmaceuticals) prior to, concurrently with, or subsequently to administration of the peptide specific CTLs.

An effective dose of peptide specific CTLs is based on body weight and can be between about 1×10⁵ total cells/kg and about 3×10⁶ total cells/kg. A dose of about 1×10⁵ total cells/kg, about 2×10⁵ total cells/kg, about 3×10⁵ total cells/kg, about 4×10⁵ total cells/kg, about 5×10⁵ total cells/kg, about 6×10⁵ total cells/kg, about 7×10⁵ total cells/kg, about 8×10⁵ total cells/kg, about 9×10⁵ total cells/kg, about 1×10⁶ total cells/kg, about 2×10⁶ total cells/kg, about 3×10⁶ total cells/kg, about 4×10⁶ total cells/kg, about 5×10⁶ total cells/kg, about 6×10⁶ total cells/kg, about 7×10⁶ total cells/kg, about 8×10⁶ total cells/kg, or about 9×10⁶ total cells/kg can be administered. In some embodiments the dose can be measured by the number of virus-reactive cells instead of the total amount. For example, an effective dose can be between about 1×10⁵ virus-reactive cells cells/kg and about 3×10⁶ virus-reactive cells/kg. A dose of about 1×10⁵ virus-reactive cells/kg, about 2×10⁵ virus-reactive cells/kg, about 3×10⁵ virus-reactive cells/kg, about 4×10⁵ virus-reactive cells/kg, about 5×10⁵ virus-reactive cells/kg, about 6×10⁵ virus-reactive cells/kg, about 7×10⁵ virus-reactive cells/kg, about 8×10⁵ virus-reactive cells/kg, about 9×10⁵ virus-reactive cells/kg, about 1×10⁶ virus-reactive cells/kg, about 2×10⁶ virus-reactive cells/kg, about 3×10⁶ virus-reactive cells/kg, about 4×10⁶ virus-reactive cells/kg, about 5×10⁶ virus-reactive cells/kg, about 6×10⁶ virus-reactive cells/kg, about 7×10⁶ virus-reactive cells/kg, about 8×10⁶ virus-reactive cells/kg, or about 9×10⁶ virus-reactive cells/kg can be administered In some embodiments the effective dose can be based on actual body weight. In certain embodiments, where the actual weight is higher than the ideal weight, the dose can be based on adjusted body weight (ideal body weight+40% the difference between actual and ideal weight). Ideal weight for height can be calculated from the formula of BJ Devine (1974): Male: 50.0 kg+2.3 kg per inch over 5 feet and Female: 45.5 kg+2.3 kg per inch over 5 feet.

An effective amount of a pharmaceutical composition comprising peptide specific CTLs can be administered to an individual in need thereof, such as an individual who has a viral infection, has a viral-like illness, is experiencing viral-like symptoms, or who is at risk for infection by an emerged strain of a virus. An effective amount is an amount that is sufficient to achieve the desired therapeutic or prophylactic effect, such as an amount sufficient to reduce viral infection, viral-like illness, or viral-like symptoms, to reduce duration of illness, to reduce virus titer, to reduce the number of days that infected individuals experience viral-like symptoms, to reduce the number of subjects who develop viral-related cytokine release syndrome, and/or to decrease the incidence or rate of viral infection. A clinician of ordinary skill can determine appropriate dosage and optionally, anti-agent, based on, for example, the individual's age, sensitivity, tolerance and overall well-being. The peptide specific CTLs can be administered in a single dose or multiple doses as indicated.

Intravenous delivery of the CTLs (e.g., infusion through a peripheral line, central line or midline) can take less than 10 minutes. In some embodiments the time to infuse the CTLs can be about 10 minutes, about 9 minutes, about 8 minutes, about 7 minutes, about 6 minutes, about 5 minutes, about 4 minutes, about 3 minutes, about 2 minutes, or about 1 minute.

In some embodiments, the methods can comprise administering an effective amount of a pharmaceutical composition to an individual suspected of having a viral infection, with a confirmed viral infection, or at risk for a viral infection. The methods can comprise administering an effective amount of a pharmaceutical composition to an individual with a viral-like illness.

The pharmaceutical compositions can be intended for administration to the blood of a subject, and can be administered in any suitable form, such as intravenously.

In some embodiments, the method can comprise administering to an individual suspected of having a viral infection or at risk of having a viral infection an effective amount of a pharmaceutical composition of the invention. For example, in some embodiments the individual can be suspected of having COVID-19 and/or can have one or more symptoms of COVID-19. Symptoms of COVID-19 are well-known and include fever, cough, and shortness of breath. Additional symptoms of COVID-19 include difficulty breathing, persistent pain or pressure in the chest, confusion, inability to arouse, bluish lips or face.

In some embodiments, the method can be for treating infection, and comprises administering to an individual in need thereof an effective amount of a pharmaceutical composition of the invention. In other embodiments, the method can be for the prophylaxis of infection and comprises administering to an individual at risk for infection by an emerged strain of a virus an effective amount of a pharmaceutical composition of the invention. In other embodiments, the method can be for reducing the spread of infection comprising administering to an individual infected by an emerged strain of a virus or at risk for infection by an emerged strain of a virus an effective amount of a pharmaceutical composition described herein.

Suitable intervals between doses that provide a desired therapeutic effect can be determined based on the severity of the condition (e.g., infection), overall well-being of the subject, the subject's tolerance to the pharmaceutical compositions, and other considerations. Based on these and other considerations, a clinician can determine appropriate intervals between doses. Generally, a pharmaceutical composition can be administered once, but may be administered every one to four days, or once a week, as needed.

After administration, successful treatment can be determined by testing subject blood and/or nasal or nasopharyngeal swab specimens for viral diagnostics, CTL persistence, the formation of endogenous CTL and antibody responses to an emerged strain of a virus. Responses to treatment may be tested, for example, at about 4 days, about 7 days, about 14 days, about 28 days, about 2 months, about 3 months, and about 6 months post-infusion.

In some embodiments, the virus can be COVID-19. In some further embodiments, an emerged strain of COVID-19 can be Delta or Omicron BA.2.75.

### Pharmaceutical Compositions

A pharmaceutical composition can comprise peptide specific CTLs prepared according to the methods disclosed herein.

The pharmaceutical compositions can be formulated for intravenous delivery to an individual in need thereof, for example, by infusion through a peripheral IV, central line, or midline catheter. The pharmaceutical compositions can also include one or more carriers or excipients that are suitable for delivery of cryopreserved CTLs, such as DMSO and the like.

In some embodiments, the pharmaceutical composition comprises specific CTLs that have been sensitized against peptides binding to specific HLA-A1 alleles.

In some embodiments, the pharmaceutical composition comprises specific CTLs that have been sensitized against peptides binding to specific HLA-A2 alleles.

In some embodiments, the pharmaceutical composition comprises specific CTLs that have been sensitized against peptides binding to specific HLA-B7 alleles.

In some embodiments, the pharmaceutical composition comprises specific CTLs that have been sensitized against peptides binding to specific HLA-B40 alleles.

In some embodiments, the pharmaceutical composition comprises specific CTLs that have been sensitized against peptides binding to specific HLA-Cw7 alleles.

In some embodiments, the pharmaceutical composition comprises specific CTLs that have been sensitized against one or more peptides binding to any one or combination of HLA-A1, -A2, -B7, -B40, and -Cw7 alleles. In another embodiment, the pharmaceutical composition comprises specific CTLs that have been sensitized against a combination of peptides binding to a combination of alleles (e.g., a half dose of -A2 CTL combined with a half dose of -B7 CTL).

In some embodiments, the pharmaceutical composition comprises cryopreserved CTLs in DMSO, RPMI-1640, albumin, or a combination thereof.

If desired, the pharmaceutical compositions described herein can also include one or more additional anti-viral agents, such as remdesivir.

The pharmaceutical composition can be in any form that is suitable for administration by a desired route, such as intravenous administration.

In some embodiments, the virus against which the peptide specific CTLs are prepared is COVID-19 and the emerged strain can be Delta or Omicron BA.2.75.

### EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compositions and methods in detail, the same will be more clearly understood by reference to the following examples, which are introduced for illustration only and not intended to be limiting.

### EXAMPLES

The following are examples of methods and compositions of the invention. It is understood that various other embodiments may be practiced, given the general description provided herein.

### Example 1. Loss of an Immunodominant HLA-A*01:01 Restricted Epitope for CD8+ Cytotoxic T Lymphocytes (CTLs) in the Delta Variant of COVID-19: An Example of Immunologic Escape and Implications for Immunologic Treatment

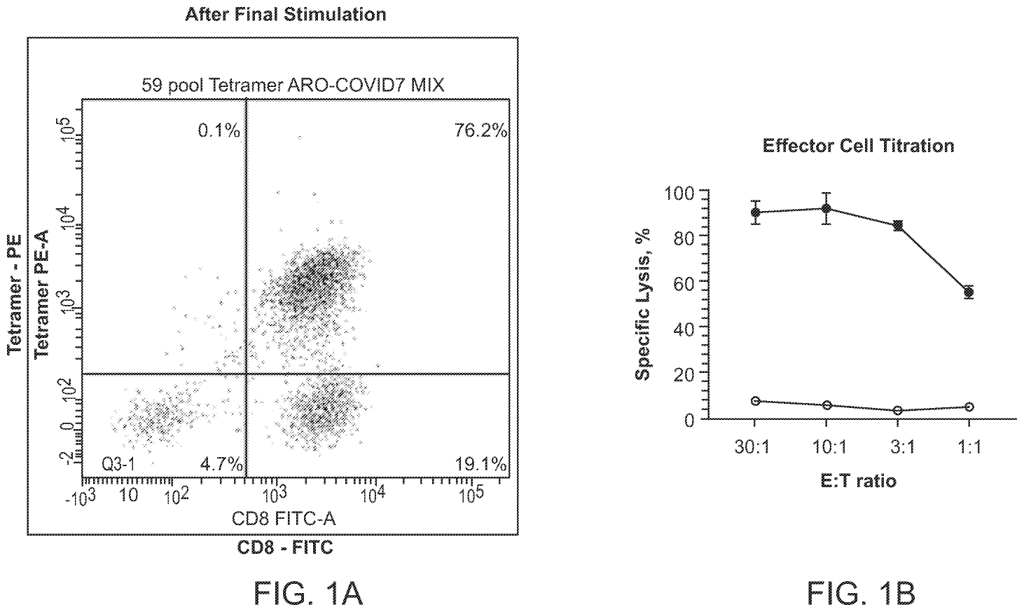

TVGN-489 is a clinical grade product consisting of highly enriched, highly potent CD8+ CTLs recognizing peptides derived from COVID-19 gene/ORF products in an HLA restricted manner. CTLs were generated from apheresis products from individuals who have recovered from COVID-19 infections. Lymphocytes were serially primed and selected using APCs from these donors pulsed with small numbers of peptides encoded by the COVID-19 genome predicted or demonstrated to bind to specific HLA class I alleles. The resulting products were typically >95% CD3+/CD8+, >60% positive by tetramer staining and demonstrated strong cytolytic activity with >60% lysis of peptide pulsed targets typically at an effector to target ratio of 3:1 (see FIGS. 1A and 1B).

Given the immunologic pressure to lose dominant target epitopes, an assessment was made whether the peptides derived from genomic sequences from early SARS-COV-2 strains (and successfully used to generate CTLs from donors infected with these early strains) were still present in the more recently evolved Delta variant.

Seven peptides were used to generate CTL products restricted by HLA-A*02:01, the most common allele worldwide. These peptides were derived from the spike(S) and nucleocapsid (N) proteins as well as ORF3a and ORF1ab. The contributions of the seven peptides to the overall cytotoxicity and tetramer staining ranged from 2% to 18% without clear immunodominance by one of these peptides (Table 1 and Table 2). Though identified in early viral strains, these sequences persisted in 97.5-100% of the more than 120 Delta variant sequences present in the NIH database.

Tetramer assays were used to quantitate viral peptide specific CD8+ T cells (in this example, CD8+ T cells sensitized against a pre-Delta strain of COVID-19) within a T cell population.

Functional analysis of virus-specific CTLs was measured as a function of CTL cytotoxicity against viral peptide-pulsed targets. Effector cells were pulsed with 2 μg/mL of viral peptide and incubated at 37° C. for a suitable amount of time. After incubation with viral peptides, the media was aspirated and effector cells were rinsed with warm PBS and fresh complete media was replaced. CTLs were titrated into wells seeded with effector cells at 30:1, 10:1, 3:1, and 1:1 ratios (non-viral peptide-pulsed effector cell conditions were kept as controls). Cytotoxicity was measured as a function of release of a radiotracer (51Cr) from cells that had undergone lysis (compared to a control in which all cells are chemically lysed). CTL-mediated cytotoxicity was observed to typically exceed 80% at E: T ratios of 30:1-3:1. Cytotoxicity was approximately 60% at effector to target ratios of 1:1.

For HLA-A*01:01, eight peptides derived from the matrix (M) protein as well as ORF1ab and ORF3a were utilized to generate CTLs. Seven of the eight peptides showed binding similar to what was seen with the HLA-A*02:01 peptides (1% to 18%) (Table 3). However, in contrast to HLA-A*02:01, an immunodominant peptide (TTDPSFLGRY, ORF1ab 1637-1646, SEQ ID NO: 1) was noted which was responsible for half of the observed tetramer binding (Table 3, Peptide 3). This region of ORF1ab was mutated in the Delta variant resulting in loss of this immunodominant epitope from nearly 93% of the Delta genomic sequences in the NIH database. The remaining subdominant peptides were all preserved in 100% of the sequences.

A similar result relating to Peptide 3 has been seen with the Omicron variant BA.2.75 (data not shown).

Given the growing number of Delta cases, it will be essential to remove this peptide from the HLA-A*01:01 peptide pool used to stimulate SARS-COV-2-specific CD8+ CTLs to avoid encouraging the expansion of cells which would recognize early strains of the virus, but not Delta variants. The remaining CTLs, generated in the absence of TTDPSFLGRY (SEQ ID NO: 1), should be capable of eradicating Delta as well as the earlier prototypic strains of COVID-19.

A similar expectation holds for the efficacy of CTLs generated in the absence of TTDPSFLGRY (SEQ ID NO: 1) against the Omicron variant BA.2.75.

The loss of immunodominant epitopes is not surprising in a virus such as SARS-COV-2, with a high frequency of mutation. This provides an example of immunologic escape similar to what has been described for the Delta variant in the case of HLA-A24. These data are consistent with the hypothesis that immunodominant epitopes will be preferentially eliminated as the virus continues to evolve. They further illustrate the need to monitor viral sequences and to tune the production of CTLs in order to ensure that they can continue to recognize and effectively treat newly emerging variants of COVID-19.

TABLE 1
Donor 5 (A02 clinical batch) run on two
different samples from clinical batch

| | | % Overall | | % Overall | Average Overall |
|---|---|---|---|---|---|
| | | Tetramer | | Tetramer | Tetramer |
| Peptide | Raw % | Staining | Raw % | Staining | Staining |
| 1 | 2.5 | 3.63% | 2.6 | 3.38% | 3.50% |
| 2 | 1.8 | 2.61% | 2 | 2.60% | 2.60% |
| 3 | 4.4 | 6.39% | 4.9 | 6.36% | 6.37% |
| 4 | 14.3 | 20.75% | 15.9 | 20.65% | 20.70% |
| 5 | 17.6 | 25.54% | 21.4 | 27.79% | 26.67% |
| 6 | 15.2 | 22.06% | 15.9 | 20.65% | 21.36% |
| 7 | 13.1 | 19.01% | 14.3 | 18.57% | 18.79% |
| Total | 68.9 | 100.00% | 77 | 100.00% | 100.00% |

TABLE 2
Donor 6 (A02) data generated from a variety of experimental
conditions GREX etc. but always after 3^{rd} stimulation

| | | | | | Average | |
|---|---|---|---|---|---|---|
| | | % Overall | | % Overall | Overall | Average of |
| | Raw | Tetramer | Raw | Tetramer | Tetramer | Donors 5 |
| Peptide | % | Staining | % | Staining | Staining | and 6* |
| 1 | 0 | 0.00% | 0.30 | 0.37% | 0.19% | 1.84% |
| 2 | 0 | 0.00% | 2.10 | 2.61% | 1.30% | 1.95% |
| 3 | 0 | 0.00% | 2.70 | 3.35% | 1.67% | 4.02% |
| 4 | 4.5 | 17.05% | 12.70 | 15.76% | 16.40% | 18.55% |
| 5 | 9.1 | 34.47% | 11.60 | 14.39% | 24.43% | 25.55% |
| 6 | 8.3 | 31.44% | 39.20 | 48.64% | 40.04% | 30.70% |
| 7 | 4.5 | 17.05% | 12.00 | 14.89% | 15.97% | 17.38% |
| Total | 26.4 | 100.00% | 80.60 | 100.00% | 100.00% | 100.00% |

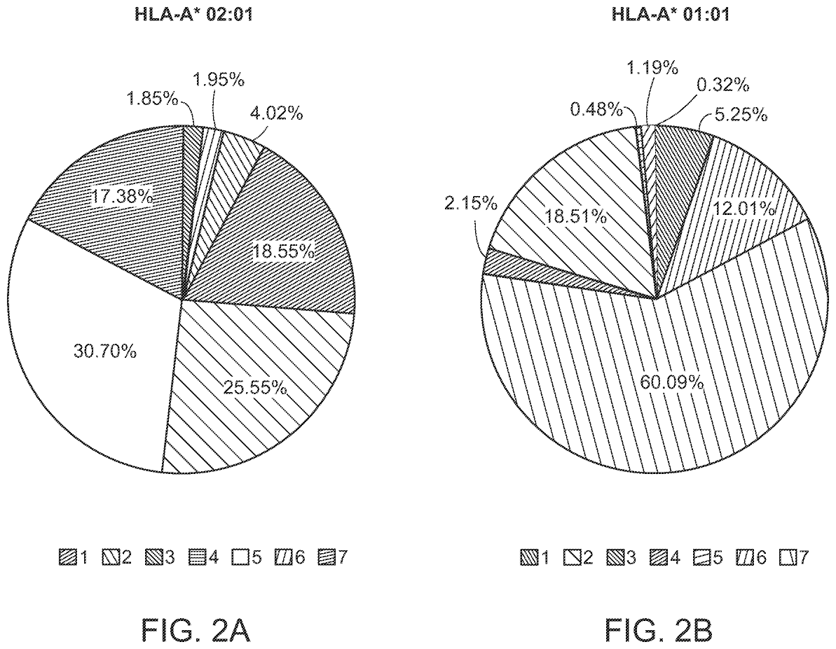

*Average of Donors 5 and 6 is depicted in FIG. 2A.

TABLE 3
Donor 7 (A01) run on two different
samples from the same experiment

| | | % Overall | | % Overall | Average Overall |
|---|---|---|---|---|---|
| | | Tetramer | | Tetramer | Tetramer |
| Peptide | Raw % | Staining | Raw % | Staining | Staining* |
| 1 | 4.1 | 4.74% | 5.7 | 5.76% | 5.25% |
| 2 | 10.9 | 12.60% | 11.3 | 11.41% | 12.01% |
| 3 | 53.8 | 62.20% | 57.4 | 57.98% | 60.09% |
| 4 | 1.1 | 1.27% | 3 | 3.03% | 2.15% |
| 5 | 14.9 | 17.23% | 19.6 | 19.80% | 18.51% |
| 6 | 0.3 | 0.35% | 0.6 | 0.61% | 0.48% |
| 7 | 1.1 | 1.27% | 1.1 | 1.11% | 1.19% |
| 8 | 0.3 | 0.35% | 0.3 | 0.30% | 0.32% |
| Total | 86.5 | 100.00% | 99 | 100.00% | 100.00% |

*Average overall tetramer staining is depicted in FIG. 2B.

### Example 2. Dilution of an Immunodominant HLA-A*01:01 Restricted Epitope

The composition containing the eight HLA-A*01:01 peptides used in Example 1 was modified by the exclusion of Peptide 8, which had negligible immunogenicity as determined by tetramer staining assay results essentially the same as background. Based on Example 1, Peptide 3 was identified as the immunodominant peptide.

Six samples were prepared, each containing the same concentration of six peptides, Peptides 1-2 and 4-7, and varying concentrations of Peptide 3 expressed as percentages of the Peptide 3 concentration in the compositions of Example 1, which was 2 μg/ml. Accordingly, the sample with 0% Peptide 3 contained six peptides, and all samples with 6.25% to 100% Peptide 3 contained seven peptides.

As shown in Table 4, the sample with 0% Peptide 3 yielded 64.1% tetramer staining. The tetramer staining value of 0.3% for Peptide 3 reflected background staining and was consistent with the sample containing 0% Peptide 3.

At 100% Peptide 3 (normal concentration, about 2 μg/ml), the total tetramer staining was roughly the same as for the 0% sample (66.8% to 64.1%), though now Peptide 3 was responsible for 25.7%. The other peptides were suppressed so that the total remains similar.

Over four serial 50% dilutions of Peptide 3, tetramer staining ranged from 74.3% to 83.1%. The contribution to total tetramer staining by Peptide 3 generally decreased as its proportion of the sample decreased.

In conclusion, the tetramer staining results presented in Table 4 indicate that reduction of the proportion of an immunodominant peptide in a peptide composition can be performed while retaining desirable levels of efficacy of the peptide composition.

TABLE 4

Tetramer %

| Sample | Mix | Peptide 3 |
|---|---|---|
| 0% Peptide 3 | 64.1% | 0.3% |
| 100% Peptide 3 | 66.8% | 25.7% |
| 50% Peptide 3 | 75.2% | 16.7% |
| 25% Peptide 3 | 74.3% | 18.6% |
| 12.5% Peptide 3 | 78.0% | 12.7% |
| 6.25% Peptide 3 | 83.1% | 7.0% |

1. A method of preparing peptide specific cytotoxic T cells (CTLs) against an emerged strain of a virus, comprising:
a. providing an initial peptide composition specific to a prior strain of the virus, against which prior CTLs were sensitized, wherein the prior CTLs have reduced efficacy against the emerged strain of the virus relative to their efficacy against the prior strain of the virus;
b identifying an immunodominant peptide against the prior strain of the virus in the initial peptide composition;
c. reducing the proportion of the immunodominant peptide in the initial peptide composition to yield an immunodominant-peptide-diluted peptide composition; and
d. sensitizing mononuclear cells with the immunodominant-peptide-diluted peptide composition, thereby producing expansion of the peptide specific CTLs against the emerged strain of the virus.

2. The method of claim 1, wherein the proportion of the immunodominant peptide in the immunodominant-peptide-diluted peptide composition is at least 30%, or less than the proportion of the immunodominant peptide in the initial peptide composition.

3. The method of claim 1, wherein the immunodominant peptide is essentially absent from the immunodominant-peptide-diluted peptide composition.

4. The method of claim 1, wherein the virus is SARS-COV-2 (COVID-19).

5. The method of claim 4, wherein the emerged strain is Delta or Omicron BA.2.75.

6. The method of claim 1, wherein the initial peptide composition comprises one or more peptides restricted against an HLA-A1 allele.

7. The method of claim 1, wherein the initial peptide composition comprises one or more peptides restricted against an HLA-A2 allele.

8. The method of claim 1, wherein the initial peptide composition comprises one or more peptides restricted against an HLA-B7 allele.

9. The method of claim 1, wherein the initial peptide composition comprises one or more peptides restricted against an HLA-B40 allele.

10. The method of claim 1, wherein the initial peptide composition comprises one or more peptides restricted against an HLA-Cw7 allele.

11. The method of claim 1, wherein the initial peptide composition comprises a combination of peptides binding to any one or more of HLA-A1, -A2, -B7, -B40, -Cw7 alleles.

12. A method of treating an emerged strain of a virus, comprising administering to a subject in need thereof an effective amount of peptide specific CTLs prepared according to the method of claim 1.

13. The method of claim 12, wherein the virus against which the peptide specific CTLs are prepared is COVID-19.

14. The method of claim 13, wherein the emerged strain is Delta or Omicron BA.2.75.

15. The method of claim 12, further comprising: administering one or more pre-medications prior to administering the CTLs.

16. The method of claim 12, further comprising: administering one or more antiviral formulations prior to, concurrently with, or subsequently to administering the CTLs.

17. A pharmaceutical composition comprising peptide specific CTLs prepared according to the method of claim 1.

18. The pharmaceutical composition of claim 17, wherein the virus against which the peptide specific CTLs are prepared is COVID-19.

19. The pharmaceutical composition of claim 18, wherein the emerged strain is Delta or Omicron BA.2.75.

20. The pharmaceutical composition of claim 17, wherein the proportion of the immunodominant peptide in the immunodominant-peptide-diluted peptide composition is at least 30%, or less than the proportion of the immunodominant peptide in the initial peptide composition, and optionally wherein the immunodominant peptide is essentially absent from the immunodominant-peptide-diluted peptide composition.

21. (canceled)
